(19) United States
(12) Patent Application Publication
(10) Pub. No.: US 2026/0207791 A1
(43) Pub. Date: Jul. 23, 2026

(54) RADIOPAQUE HYDROGELS AND METHODS OF MAKING AND USING SAME

(71) Applicant: Boston Scientific Scimed, Inc., Maple Grove, MN (US)

(72) Inventors: Joseph Thomas Delaney, JR., Minneapolis, MN (US); Rick William Dorn, Plymouth, MN (US); Cristian Parisi, Amherst, MA (US); Nicolas Ball-Jones, Minneapolis, MN (US)

(73) Assignee: Boston Scientific Scimed, Inc., Maple Grove, MN (US)

(21) Appl. No.: 19/453,341

(22) Filed: Jan. 20, 2026

Related U.S. Application Data
(60) Provisional application No. 63/747,483, filed on Jan. 21, 2025.

(51) Int. Cl. A61K 49/04

(52) U.S. Cl. CPC A61K 49/0442

(57) ABSTRACT

In some aspects, the present disclosure pertains to systems that comprise: an iodinated multi-arm polymer comprising first hydrophilic polymer segments and iodinated end groups, the iodinated multi-arm polymer comprising a first core region and three or more polymer arms linked to the first core region, each of the polymer arms comprising a first hydrophilic polymer segment and an iodinated end group; a reactive multi-arm polymer comprising second hydrophilic polymer segments and first reactive groups, the reactive multi-arm polymer comprising a second core region and three or more polymer arms linked to the second core region, each of the polymer arms comprising a second hydrophilic polymer segment and a first reactive group; and a reactive multifunctional compound comprising a plurality of second reactive groups, the second reactive groups forming covalent bonds with the first reactive groups. Methods of using such systems and crosslinked products of such systems are also described.

### CROSS-REFERENCE TO RELATED APPLICATIONS

This application claims the benefit of U.S. Provisional Patent Application Ser. No. 63/747,483 filed on Jan. 21, 2025, the disclosure of which is incorporated herein by reference.

### FIELD

The present disclosure relates to radiopaque hydrogels, to crosslinkable systems for forming such radiopaque hydrolysable hydrogels, and to methods of treatment using such radiopaque hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

The present disclosure provides implantable hydrogel alternatives to SpaceOAR Vue®, thereby expanding the range of medical applications for the hydrogels.

### SUMMARY

In some aspects, the present disclosure pertains to systems that comprise (a) an iodinated multi-arm polymer comprising first hydrophilic polymer segments and iodinated end groups, the iodinated multi-arm polymer comprising a first core region and three or more polymer arms linked to the first core region, each of the polymer arms comprising a first hydrophilic polymer segment and an iodinated end group, (b) a reactive multi-arm polymer comprising second hydrophilic polymer segments and first reactive groups, the reactive multi-arm polymer comprising a second core region and three or more polymer arms linked to the second core region, each of the polymer arms comprising a second hydrophilic polymer segment and a first reactive group and (c) a reactive multifunctional compound comprising a plurality of second reactive groups, the second reactive groups forming covalent bonds with the first reactive groups.

In some embodiments, the iodinated end group comprises at least one iodine-substituted monocyclic or multicyclic aromatic group.

In some embodiments, which can be used in conjunction with the above aspects and embodiments, the iodine-substituted monocyclic or multicyclic aromatic group is substituted with one, two, three, four, five, six or more iodine atoms.

In some embodiments, which can be used in conjunction with the above embodiments, the iodine-substituted monocyclic or multicyclic aromatic group is further substituted with one or more hydrophilic groups that are directly linked to the iodine-substituted monocyclic or multicyclic aromatic group or are linked to the iodine-substituted monocyclic or multicyclic aromatic group through a linking moiety that comprises amide group, an ether group, ester group, a carbamate group or a combination thereof. In some of these embodiments, the hydrophilic groups are selected from acetamido groups and hydroxyl-containing groups.

In some embodiments, which can be used in conjunction with the above embodiments, the iodine-substituted monocyclic or multicyclic aromatic group is linked to the first hydrophilic segment through a linkage that comprises an amide group, an ether group, an ester group, a carbamate groups or a combination thereof. In some of these embodiments, the iodine-substituted monocyclic or multicyclic aromatic group is linked to the first hydrophilic segment through a linkage that comprises an amide group and an ester group. In some of these embodiments, the iodine-substituted monocyclic or multicyclic aromatic group is linked to the first hydrophilic segment through a linkage that comprises two ester groups. In some of these embodiments, the iodine-substituted monocyclic or multicyclic aromatic group is linked to the first hydrophilic segment through a linkage that comprises an amide group and a carbamate group. In some of these embodiments, the iodine-substituted monocyclic or multicyclic aromatic group is linked to the first hydrophilic segment through a linkage that comprises an amide group and an ether group.

In some embodiments, which can be used in conjunction with the above aspects and embodiments, the first core region, the second core region, or the first and second core regions comprise a polyol residue. In some of these embodiments, the first hydrophilic polymer segments are linked to the first core region through a hydrolysable ester.

In some embodiments, which can be used in conjunction with the above aspects and embodiments, the first hydrophilic polymer segments, the second hydrophilic polymer segments, or both the first and second hydrophilic polymer segments are selected from poly(alkylene oxide) segments, polysaccharide segments, polyoxazoline segments, polydioxanone segments, polypeptide segments, and polyvinyl alcohol segments.

In some embodiments, which can be used in conjunction with the above aspects and embodiments, each of the first hydrophilic polymer segments, each of the second hydrophilic polymer segments, or each of the first and second the hydrophilic polymer segments contains between 10 and 1000 monomer residues.

In some embodiments, which can be used in conjunction with the above aspects and embodiments, the first reactive end groups are linked to the second hydrophilic polymer segments through a linkage that comprises a hydrolysable ester or linkage that does not comprise a hydrolysable ester.

In some embodiments, which can be used in conjunction with the above aspects and embodiments, the first reactive end groups are electrophilic groups. In some of these embodiments, the electrophilic groups are cyclic imide groups.

In some embodiments, which can be used in conjunction with the above aspects and embodiments, the second reactive groups are nucleophilic groups. In some of these embodiments, the nucleophilic groups are primary amine groups.

In some embodiments, which can be used in conjunction with the above aspects and embodiments, the system further comprises a delivery device.

In other aspects, the present disclosure pertains to methods treatment comprising administering to a subject a mixture that comprises a reactive multi-arm polymer in accordance with of any of the above aspects and embodiments, a reactive multifunctional compound in accordance with any of the above aspects and embodiments, and an iodinated multi-functional polymer in accordance with any of the above aspects and embodiments, under conditions such that the reactive multi-arm polymer and the reactive multifunctional compound crosslink after administration to form a hydrogel in which the iodinated multi-functional polymer is retained in the hydrogel via non-covalent interactions.

In some embodiments, the method comprises administering to the subject a first fluid composition that comprises the reactive multi-arm polymer and a second fluid composition that comprises the reactive multifunctional compound, wherein the iodinated multi-functional polymer is provided in the first fluid composition, the second composition, or both.

In some embodiments, the method comprises administering to the subject a first fluid composition that comprises the reactive multi-arm polymer and the reactive multifunctional compound and a second fluid composition that comprises an accelerant that accelerates formation of the covalent crosslinks, wherein the iodinated multi-functional polymer is provided in the first fluid composition, the second composition, or both.

In some embodiments, the first fluid composition and the second fluid composition are delivered using a double barrel syringe.

In further aspects, the present disclosure pertains to radiopaque crosslinked hydrogel compositions wherein an iodinated multi-functional polymer in accordance with of any of the above aspects and embodiments is retained in a crosslinked reaction product of a reactive multi-arm polymer in accordance with of any of the above aspects and embodiments and a reactive multifunctional compound in accordance with of any of the above aspects and embodiments.

In some of the embodiments, the radiopaque crosslinked hydrogel compositions are in the form of an injectable particle.

In additional aspects, the present disclosure pertains to methods of treatment comprising administering to a subject a radiopaque crosslinked hydrogel composition in accordance with of any of the above aspects and embodiments.

Potential benefits associated with the present disclosure include one or more of the following: overall level of radiocontrast is maintained or enhanced, crosslink density is tunable independently of radiocontrast, and in vivo radiocontrast persistence is obtained, particularly relative to the use of nonpolymeric contrast agents such as iodixanol and ioxanol among many others.

The above and other aspects, embodiments, features and benefits of the present disclosure will be readily apparent from the following detailed description.

### BRIEF DESCRIPTION OF THE DRAWINGS

FIG. 1 schematically illustrates the formation of a hydroxyl-terminated 8-arm-polyethylene oxide having a tripentaerythritol residue core, in accordance with an embodiment of the present disclosure.

FIG. 2 schematically illustrates the formation of a hydroxyl-terminated 8-arm-polyoxazoline having a tripentaerythritol residue core, in accordance with an embodiment of the present disclosure.

FIG. 3 schematically illustrates the formation of a hydroxyl-terminated 3-arm-polyethylene oxide having a 1,3,5-triiodo-2,4,6-tris-hydroxymethylbenzene residue core, in accordance with an embodiment of the present disclosure.

FIG. 4 schematically illustrates the formation of an amino-terminated multi-arm-polyethylene oxide, in accordance with an embodiment of the present disclosure.

FIG. 5 schematically illustrates the formation of a glutaric-acid-ester-terminated multi-arm-polyethylene oxide, in accordance with an embodiment of the present disclosure.

FIG. 6 schematically illustrates the formation of a diatrizoic-acid-ester-terminated polymer arm, in accordance with an embodiment of the present disclosure.

FIG. 7 schematically illustrates a process in which thyroxine methyl ester is coupled to a glutaric acid ester end group of a polymer arm in an amide coupling reaction, in accordance with an embodiment of the present disclosure.

FIG. 8 schematically illustrates a process in which acetal-protected iodixanol is coupled to a glutaric acid ester end group of a polymer arm in an ester coupling reaction, in accordance with an embodiment of the present disclosure.

FIG. 9 schematically illustrates a process in which iodixanol is reacted with a CBZ-protected isocyanate-functionalized amine, followed by Cbz deprotection, to form an iodixanol derivative in which an amino-alkyl group is linked to an iodixanol residue through a carbamate group, in accordance with an embodiment of the present disclosure.

FIG. 10 schematically illustrates a method in which iodixanol is reacted Boc-protected 3-bromo-propylamine in the presence of potassium carbonate, followed by removal of the Boc protection, to yield an amino-functionalized iodixanol derivative in which an amino-alkyl group is linked to an iodixanol residue through an ether group, in accordance with an embodiment of the present disclosure.

FIG. 11A schematically illustrates a method in which acetal-protected iopamidol is reacted with Boc-protected 1,5-dibromo-3-aminopentane, followed by removal of the Boc protection, to form an amino-functionalized iodinated compound in which an amino-alkyl group is linked to two iopamidol residues through ether groups, in accordance with an embodiment of the present disclosure.

FIG. 11B schematically illustrates a method in which Boc-protected 3-aminopentane, 1,5-dicarboxylic acid treated with sodium borohydride to form Boc-protected 1,5-dihyoxy-3-aminopentane, which is then reacted with triphenylphosphine and carbon tetrabromide to form Boc-protected 1,5-dibromo-3-aminopentane, in accordance with an embodiment of the present disclosure.

FIG. 12 schematically illustrates a method of forming succinimidyl-glutarate-terminated multi-arm polyethylene oxide, in accordance with an embodiment of the present disclosure.

FIG. 13 schematically illustrates a delivery device, in accordance with an embodiment of the present disclosure.

FIG. 14 schematically illustrates a delivery device, in accordance with another embodiment of the present disclosure.

### DETAILED DESCRIPTION

In some aspects, the present disclosure provides iodinated multi-arm polymers that comprise a core region and three or more polymer arms extending from the core region, each of the polymer arms comprising a hydrophilic polymer segment and an iodinated end group. As discussed further below, such iodinated multi-arm polymers are useful, for example, as contrast agents for crosslinked hydrogel systems.

The iodinated end group may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others.

Iodinated multi-arm polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, ranging between any two of the preceding values).

Hydrophilic polymer segments for the polymer arms of the iodinated multi-arm polymers can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments for use in the iodinated multi-arm polymers of the present disclosure may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Hydrophilic polymer segments for use in the multi-arm polymers of the present disclosure typically contain between 10 and 1000 monomer units or more, for example ranging from 10 to 25 to 50 to 100 to 250 to 500 to 1000 monomer units.

Iodinated end groups for use in the multi-arm polymers of the present disclosure include those that contain one or more iodinated aromatic groups. Examples of iodinated aromatic groups include iodine-substituted monocyclic aromatic groups and iodine-substituted multicyclic aromatic groups, such as iodine-substituted benzene groups, iodine-substituted naphthylene groups, iodine-substituted anthracene groups, iodine-substituted phenanthrene groups and iodine-substituted tetracene groups, among others. The iodinated aromatic groups may be substituted with one, two, three, four, five, six or more iodine atoms. In some embodiments, the iodinated aromatic groups are further substituted with one or more hydrophilic groups, which may be linked to the aromatic groups directly or may be linked to the aromatic groups through a suitable linking moiety, which may comprise, for example, an amide group, an ether group, and ester group, a carbamate group or a combination thereof, among others. Hydrophilic groups include, for example, acetamido groups that are directedly linked to the aromatic groups, hydroxyl-containing groups including hydroxyl groups that are directly linked to the aromatic groups and hydroxyalkyl groups (e.g., C₁-C₄-hydroxyalkyl groups containing one, two, three or four carbon atoms and containing one, two, three or four or more hydroxyl groups) that are directly linked to the aromatic groups or linked to the aromatic groups through a suitable linking moiety such as those described above.

Core regions for use in the present disclosure include core regions that comprise a residue of a polyhydroxy compound comprising three or more hydroxyl groups, also referred to herein as a “polyol”, which is used to form the polymer arms. In certain beneficial embodiments, the core regions comprise residues of non-iodinated or iodinated polyols that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

In some embodiments of the present disclosure, a non-iodinated polyol or an iodinated polyol such as one of those described below, among others, may be used to form a multi-functional initiator for polymer chain growth.

For example, a non-iodinated or iodinated polyol may be used as an initiator for ring-opening polymerization of ethylene oxide to form polyethylene oxide (PEO) segments (also referred to a polyethylene glycol (PEG) segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity in some embodiments.

In a particular embodiment shown in FIG. 1, tripentaerythritol (110) is used as an octa-functional initiator, which undergoes ring-opening polymerization with ethylene oxide (111). The polymerization process leads to poly(ethylene oxide) chain growth at each of the eight hydroxyl groups of the tripentaerythritol, forming an 8-arm-PEG (112) having a tripentaerythritol residue core. Each of the PEG arms has a terminal hydroxyl group. In FIG. 1, n is an integer representing the number of monomer units in each polymer segment shown.

As another example, a non-iodinated or iodinated polyol may be reacted with methanesulfonyl chloride to convert the hydroxyl groups into methanesulfonate groups, which can serve as leaving groups for the cationic polymerization of an oxazoline monomer to form polyoxazoline segments at the site of each of the hydroxyl groups of the polyol. The resulting hydroxyl-terminated polyoxazoline segments possess tunable hydrophilicity depending on the desired water-solubility of the resulting multi-arm polymer, for example, with increasing polyoxazoline segment length leading to increased hydrophilicity in some embodiments.

In a particular embodiment shown in FIG. 2, tripentaerythritol (210) is reacted with methanesulfonyl chloride (212) to convert the hydroxyl groups of the tripentaerythritol into methanesulfonate groups, thereby forming an octa-functional initiator (214), which can be used for the polymerization of 2-ethyl-2-oxazoline (216). The polymerization process leads to poly(2-ethyl-2-oxazoline) chain growth at sites previously occupied by each of the eight hydroxyl groups of the tripentaerythritol, forming a hydroxyl-terminated 8-arm-poly(2-ethyl-2-oxazoline) (218) having a tripentaerythritol residue core and eight poly(2-ethyl-2-oxazoline) arms. Each of the polyoxazoline arms has a terminal hydroxyl group. In FIG. 2, n is an integer representing the number of monomer units in the polymer segment shown. Only a single polymer arm of the hydroxyl-terminated 8-arm-poly(2-ethyl-2-oxazoline) (218) is shown, with R representing the core and the remaining seven arms of the hydroxyl-terminated 8-arm-poly(2-ethyl-2-oxazoline) (218).

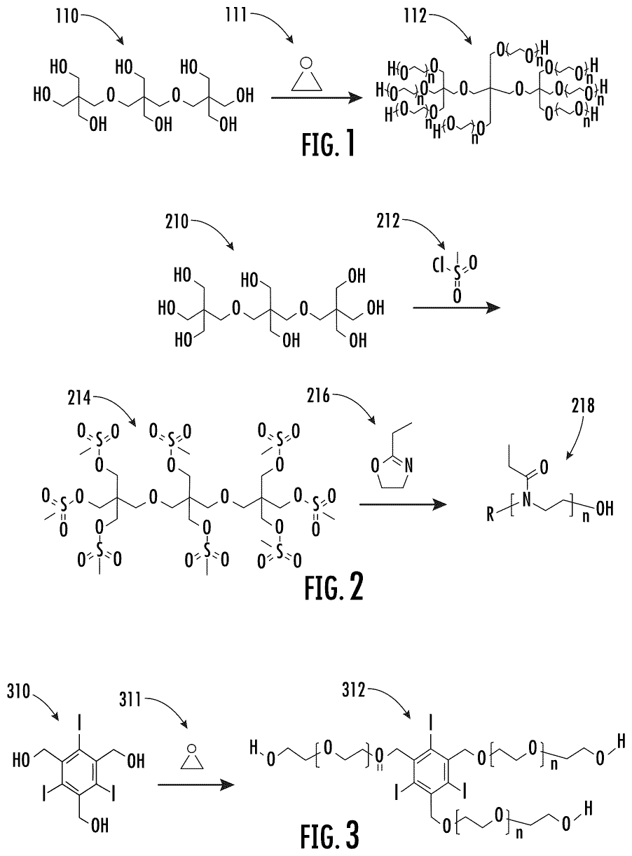

The strategies shown in FIGS. 1 and 2 can be used in conjunction with a wide range of polyols, including those described below. In another particular embodiment shown in FIG. 3, an iodinated polyol, specifically, 1,3,5-triiodo-2,4,6-tris-hydroxymethylbenzene (310), is used as an initiator, which undergoes ring-opening polymerization with ethylene oxide (311). The polymerization process leads to poly(ethylene oxide) (PEG) chain growth at each of the three hydroxyl groups of the 1,3,5-triiodo-2,4,6-tris-hydroxymethylbenzene (310). The resulting multi-arm polymer (312) contains three PEG arms that extend from a 1,3,5-triiodo-2,4,6-tris-hydroxymethylbenzene residue core. Each of the PEG arms has a terminal hydroxyl group. In FIG. 3, n is an integer representing the number of monomer units in each polymer segment shown.

Illustrative non-iodinated polyols may be selected, for example, from sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Illustrative non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative non-iodinated polyols also include polyhydroxylated polymers. For example, in some embodiments, the core region comprises a polyhydroxylated polymer residue such as a poly(vinyl alcohol) residue, a poly(allyl alcohol) residue, a polyhydroxyethyl acrylate residue, or a polyhydroxyethyl methacrylate residue, among others. Such polyhydroxylated polymer residues may range, for example, from 3 to 100 monomer units in length.

Iodinated polyols are desirable where additional radiopacity is desired. Illustrative iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl-containing groups, including hydroxyl groups that are directly linked to the aromatic groups and hydroxyalkyl groups (e.g., C₁-C₄-hydroxyalkyl groups containing one, two, three or four carbon atoms and containing one, two, three or four or more hydroxyl groups), which may be linked to the aromatic group directly or through any suitable linking moiety, which may be selected, for example, from amide groups, ester groups, ether groups, carbamate groups, and combinations thereof, among others.

Further illustrative iodinated polyols for use in the present disclosure, in addition to the 1,3,5-triiodo-2,4,6-tris-hydroxymethylbenzene described above, include iodinated polyols that are known for use as iodinated contrast agents, whose biocompatibility has been demonstrated to be reasonably well tolerated. Some specific examples of non-iodinated and iodinated polyols are listed in the following table.

| | | # of OH |
|---|---|---|
| Species | CAS | Groups |
| 2-(Hydroxymethyl)-1,3-propanediol | 4704-94-3 | 3 |
| Glycerol | 58-81-5 | 3 |
| Trimethylolethane | 77-85-0 | 3 |
| Trimethylolpropane | 77-99-6 | 3 |
| Diglycerol | 9627-82-7 | 4 |
| Erythritol | 149-32-6 | 4 |
| Pentaerythritol | 115-77-5 | 4 |
| Iopromide* | 73334-07-3 | 4 |
| 5-Acetamido-N,N′-bis(2,3- | 31127-80-7 | 4 |
| dihydroxypropyl)-2,4,6- | | |
| triiodoisophthalamide* | | |
| Metrizamide* | 31112-62-6 | 4 |
| 1,3-Benzenedicarboxamide, 5- | 76350-28-2 | 4 |
| (acetylamino)-N1,N3-bis(2,3- | | |
| dihydroxypropyl)-2,4,6-triiodo-N1- | | |
| methyl- (ACI)* | | |
| Arabitol | 448-82-4 | 5 |
| Triglycerol | 20411-31-8 | 5 |
| Xylitol | 87-99-0 | 5 |
| Iopamidol* | 60166-93-0 | 5 |
| Iomeprol* | 78649-41-9 | 5 |
| Iotrolan*, 1,3-Benzenedicarboxamide, | 60208-45-9 | 5 |
| N1,N3-bis[2-hydroxy-1- | | |
| (hydroxymethyl)ethyl]-5-[(2-hydroxy-1- | | |
| oxopropyl)amino]-2,4,6-triiodo- (ACI)* | | |
| Ioxilan* | 107793-72-6 | 5 |
| Iopentol* | 89797-00-2 | 5 |
| Ioversol* | 87771-40-2 | 6 |
| Dipentaerythritol | 126-58-9 | 6 |
| Iohexol* | 66108-95-0 | 6 |
| Mannitol | 69-65-8 | 6 |
| Sorbitol | 50-70-4 | 6 |
| Iobitridol* | 136949-58-1 | 6 |
| Octa-(3-hydroxypropyl) POSS | 229967-81-1 | 8 |
| Tripentaerythritol | 78-24-0 | 8 |
| Iodixanol* | 92339-11-2 | 9 |
| Isomalt | 64519-82-0 | 9 |
| Maltitol | 585-88-6 | 9 |
| 1,3-Benzenedicarboxamide, 5,5′-[(1,3- | 79770-24-4 | 12 |
| dioxo-1,3- | | |
| propanediyl)bis(methylimino)]bis[N,N′- | | |
| bis[2,3-dihydroxy-1- | | |
| (hydroxymethyl)propyl]-2,4,6-triiodo- | | |
| (9CI, ACI)* | | |

*Iodinated Compounds.

Iodinated multi-arm polymers in accordance with the present disclosure can be formed from hydroxyl-terminated multi-arm polymers through various synthetic routes.

In some embodiments, for example, where it is desired to form an iodinated multi-arm polymer in which the arms can be released from the core by hydrolysis in vivo, hydroxyl groups of a polyol such as one of those described above, can be reacted with a cyclic anhydride compound (e.g., glutaric anhydride, succinic anhydride, malonic anhydride, adipic anhydride, diglycolic anhydride, 1,3-acetonedicarboxylic acid anhydride, etc.) to form a polycarboxylic acid compound in which each carboxylic acid group is attached to a polyol residue through a hydrolysable ester group. (Additional cyclic anhydrides, some of which are some of which are iodinated, are listed below.) The carboxylic acid groups of the polycarboxylic acid compound may then be reduced to hydroxyl groups using a suitable reducing agent such as borane complexes (which comprise —BH₃ groups) thereby forming a hydrolysable polyol compound in which each hydroxyl group is attached to the polyol residue through a hydrolysable ester group. This hydrolysable polyol compound can then be used in the synthesis of polymer arms in the same way as the iodinated and non-iodinated polyols described above.

In some embodiments, hydroxyl end groups of a hydroxyl-terminated multi-arm polymer are converted into amino groups, thereby forming amino-terminated multi-arm polymer having arms that each comprise one or more amino end groups.

For example, a multi-arm polymer that comprises a core region and a plurality of polymer arms with hydroxyalkyl end groups may be reacted with methanesulfonyl chloride to form a multi-arm polymer that comprises a core region and a plurality of polymer arms having methanesulfonate end groups. The methanesulfonate end groups may then be reacted with ammonia to form a multi-arm polymer that comprises a core region and a plurality of polymer arms with amino end groups.

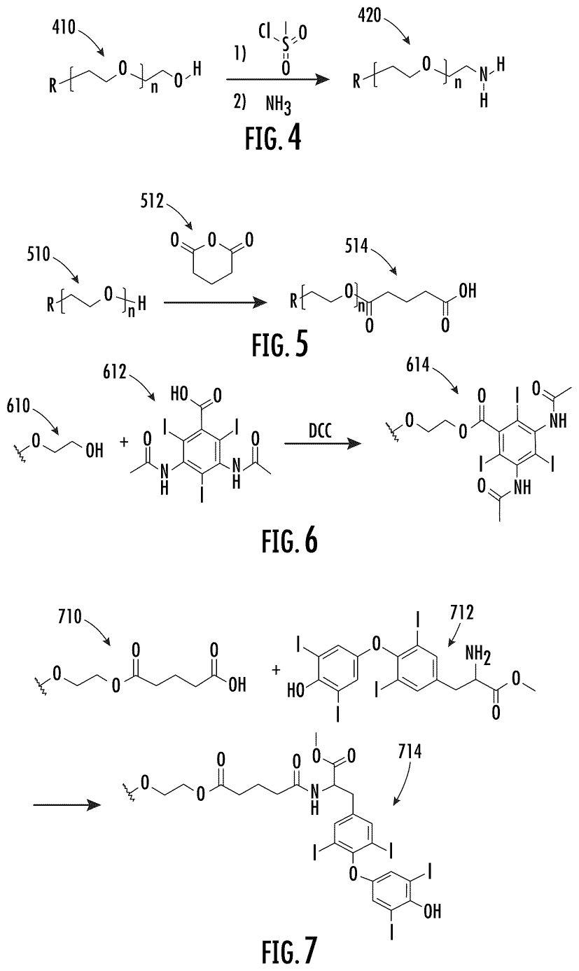

In a particular example shown in FIG. 4, a hydroxyl-terminated multi-arm PEG (410) having a polyol core, where n represents the number monomer units (note that only one polymer arm is shown; the polyol residue and the remaining arms are represented by R), for example, a hydroxyl-terminated 8-arm PEG (410) like that of FIG. 1, where R may comprise, for example, a tripentaerythritol residue and n ranges from 30 to 140, is first treated with methanesulfonyl chloride to form a polymer in which hydroxyl end groups of the 8-arm PEG (410) are converted into methanesulfonate groups. The methanesulfonate groups are then reacted with ammonia to convert the methanesulfonate groups to amino groups, thereby forming an amino-terminated multi-arm, specifically, an amino-terminated 8-arm PEG (420). Analogous processes can be performed with other hydroxyl-terminated multi-arm polymers, including those of FIGS. 2 and 3, among many others.

Various amino-terminated multi-arm polymers are also available commercially. For example, amino-terminated three-arm PEG,

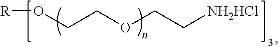

where R represents a glycerol residue core, amino-terminated four-arm PEG,

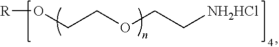

where R represents a pentaerythritol residue core, amino-terminated six-arm PEG,

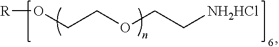

where R represents a dipentaerythritol residue core, and amino-terminated eight-arm PEG,

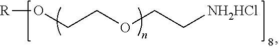

where R represents a tripentaerythritol residue core, are available from JenKem Technology USA, Plano, TX, USA. The preceding amino-terminated multi-arm polymers are shown in salt form, specifically, in the hydrochloride salt form.

In some embodiments, a hydroxyl-terminated multi-arm polymer is converted into a carboxylic-acid-terminated multi-arm polymer having arms that each comprise a carboxylic acid end group.

In particular embodiments, terminal hydroxyl groups of a hydroxyl-terminated multi-arm polymer may be reacted with a cyclic anhydride compound (e.g., glutaric anhydride, succinic anhydride, malonic anhydride, adipic anhydride, diglycolic anhydride, 1,3-acetonedicarboxylic acid anhydride, etc.) to form carboxylic-acid-terminated polymer arms such as glutaric-acid-ester-terminated polymer arms, succinic-acid-ester-terminated polymer arms, malonic-acid-ester-terminated polymer arms, adipic-acid-ester-terminated polymer arms, diglycolic-acid-ester-terminated polymer arms, 1,3-acetonedicarboxylic-acid-ester-terminated polymer arms, and so forth. Various anhydrides, some of which are iodinated, are listed in the following table. The use of such iodinated anhydrides will result in additional radiopacity for the iodinated multi-arm polymers form using the same.

| Species | CAS # |
|---|---|
| Succinic Anhydride | 108-30-5 |
| Glutaric Anhydride | 108-55-4 |
| Adipic Anhydride | 2035-75-8 |
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| Diglycolic anhydride | 4480-83-5 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |
| Dihydro-3-(2-iodophenyl)-2,5-furandione* | 887131-98-8 |

*Iodinated Compounds.

The preceding cyclic anhydrides, among others, may be reacted with a hydroxy-terminated multi-arm polymer under basic conditions to form a carboxylic-acid-terminated multi-arm polymer in which the polymer arms comprise a carboxylic acid end group that is linked to the polymer arm through a hydrolysable ester group.

In a particular example shown in FIG. 5, a hydroxyl-terminated multi-arm polymer, for example, a hydroxyl-terminated 8-arm PEG (514) like that of FIG. 1, where n is an integer representing the number of monomer units in the polymer segment shown and R represents a remainder of the molecule (including the polyol residue core and the remaining arms of the molecule) is reacted with glutaric anhydride (512) to yield a glutaric-acid-ester-terminated multi-arm polymer, specifically, a glutaric-acid-ester-terminated 8-arm PEG (514).

Various carboxyl-terminated multi-arm polymers are also available commercially. For example, carboxyl-terminated four-arm PEG,

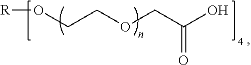

where n is an integer and R represents a pentaerythritol residue core, carboxyl-terminated eight-arm PEG,

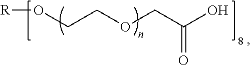

where R represents a hexaclycerol residue core, are available from JenKem Technology USA, Plano, TX, USA. Unlike anhydride-based, carboxyl-terminated multi-arm polymers described above, these carboxyl-terminated multi-arm polymers do not contain hydrolysable ester linkages.

After creating or obtaining a multi-arm polymer with the desired reactive end groups, these end groups can then be reacted with a suitable iodinated compound to provide a variety of iodinated multi-arm polymers in accordance with the present disclosure, which comprise a core region and three or more polymer arms attached to the core region, the polymer arms each having an iodinated end group. In some embodiments the iodinated compound is a water-soluble aromatic iodinated compound.

In some embodiments, a carboxylic-acid-containing iodinated compound is coupled to a multi-arm polymer with alcohol end groups via an ester coupling reaction. Such reactions may be performed in the presence of a suitable ester coupling agent. Examples of suitable ester coupling agents include carbodiimide coupling agents such as N,N′-dicyclohexylcarbodiimide (DCC), 1-ethyl-3-(3-dimethyl′propyl)carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others. In a particular embodiment shown in FIG. 6, diatrizoic acid (612), also known as 3,5-diacetamido-2,4,6-triiodo-benzoic acid, is coupled to a multi-arm polymer with hydroxyl end groups (610) via an ester coupling reaction in the presence of N,N′-dicyclohexylcarbodiimide (DCC) as an ester coupling agent to form a multi-arm polymer (614) having 3,5-diacetamido-2,4,6-triiodo-benzoate end groups.

In some embodiments, a carboxylic acid containing iodinated species is coupled to a multi-arm polymer with amine end groups via an amide coupling reaction. Such reactions may be performed in the presence of a suitable amide coupling agent. Examples of suitable amide coupling agents include carbodiimide coupling agents such as N,N′-dicyclohexylcarbodiimide (DCC), 1-ethyl-3-(3-dimethyl′propyl)carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others. In a particular embodiment, diatrizoic acid may be coupled to a multi-arm polymer with amino end groups via an amide coupling reaction in the presence of N,N′-dicyclohexylcarbodiimide (DCC) as an amide coupling agent to form a multi-arm polymer having 3,5-diacetamido-2,4,6-triiodo-benzamido end groups.

Examples of carboxylic-acid-containing iodinated compounds include those that comprise at least one iodinated aromatic group (e.g., a monocyclic or multicyclic aromatic structure that is substituted with one, two, three, four, five, six or more iodine atoms) and a carboxylic acid group. Specific examples of carboxylic-acid-containing iodinated molecule include the following, among many others: hydroxymethyl iodobenzoic acid,

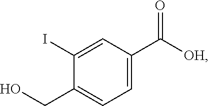 [SMILES: O=C(O)c1ccc(CO)c(I)c1]

hydroxymethyl diiodobenzoic acid,

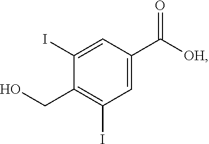 [SMILES: O=C(O)c1cc(I)c(CO)c(I)c1]

4, 4-bis(hydroxy-3, 5-diiodophenyl) pentanoic acid (IBHP),

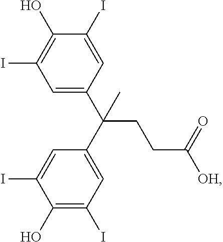 [SMILES: CC(CCC(=O)O)(c1cc(I)c(O)c(I)c1)c1cc(I)c(O)c(I)c1]

triiodobenzoic acid,

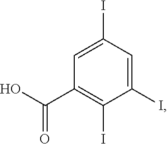 [SMILES: O=C(O)c1cc(I)cc(I)c1I]

and diatrizoic acid,

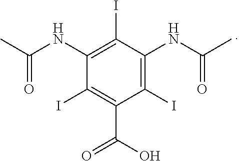 [SMILES: CC(=O)Nc1c(I)c(NC(C)=O)c(I)c(C(=O)O)c1I]

In some embodiments the carboxylic-acid-containing iodinated species is an N-acetyl derivative of an iodinated amino acid, examples of which include N-acetyl-3,5-diiodo-L-tyrosine,

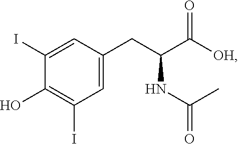 [SMILES: CC(=O)N[C@@H](Cc1cc(I)c(O)c(I)c1)C(=O)O]

N-acetyl-3-diiodo-L-tyrosine,

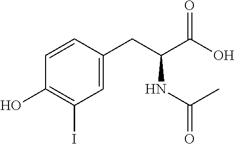 [SMILES: CC(=O)N[C@@H](Cc1ccc(O)c(I)c1)C(=O)O]

and N-acetyl-thyroxine,

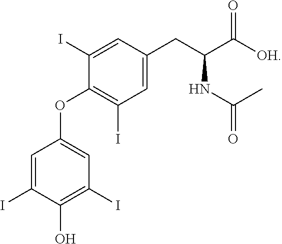 [SMILES: CC(=O)N[C@@H](Cc1cc(I)c(Oc2cc(I)c(O)c(I)c2)c(I)c1)C(=O)O]

Various carboxylic-acid-containing iodinated molecules, along with their CAS numbers, are listed in the following table:

| Name | CAS | # I atoms |
|---|---|---|
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid* | 79211-41-9 | 3 |
| 2,4,6-Triiodobenzoic acid | 2012-31-9 | 3 |
| Benzoic acid, 3-[[[2-hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, (S)- | | |
| (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid* | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid* | 2618-25-9 | 6 |
| Iocarmic acid* | 10397-75-8 | 6 |

For iodinated species that have more than one carboxylic acid functional group, a significant molar excess of the iodinated species can be used during the coupling reactions ensure that only one of the carboxyl groups of the iodinated species is attached to a polymer arm for a majority of the reacted iodinated species. The product can be further purified via chromatography or fractional crystallization methods.

In some embodiments, an amine containing iodinated species is coupled to a multi-arm polymer with carboxylic acid end groups via an amide coupling reaction. Such reactions may be performed in the presence of a suitable amide coupling agent. Examples of suitable amide coupling agents include carbodiimide coupling agents such as those listed above. In some embodiments the amine containing iodinated species is an alkyl ester of an iodinated amino acid. Specific examples of iodinated amino acid esters include the following: monoiodo-phenylalanine methyl ester,

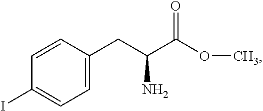 [SMILES: COC(=O)[C@@H](N)Cc1ccc(I)cc1]

monoiodotyrosine methyl ester,

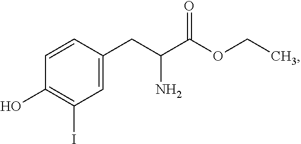 [SMILES: CCOC(=O)C(N)Cc1ccc(O)c(I)c1]

diiodotyrosine methyl ester,

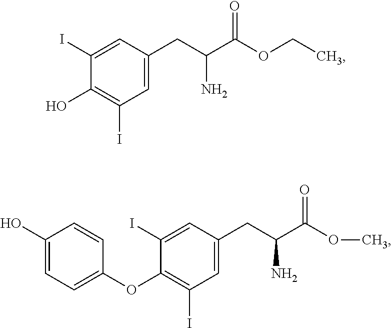 [SMILES: CCOC(=O)C(N)Cc1cc(I)c(O)c(I)c1 ; COC(=O)[C@@H](N)Cc1cc(I)c(Oc2ccc(O)cc2)c(I)c1]

triiodothyronine methyl ester, also known as T3 methyl ester,

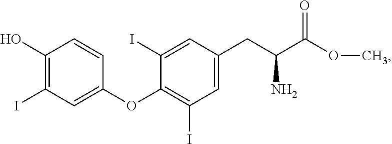 [SMILES: COC(=O)[C@@H](N)Cc1cc(I)c(Oc2ccc(O)c(I)c2)c(I)c1]

tetraiodothyronine methyl ester, also known as thyroxine methyl ester or T4 methyl ester,

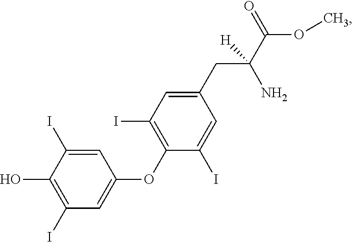 [SMILES: COC(=O)[C@@H](N)Cc1cc(I)c(Oc2cc(I)c(O)c(I)c2)c(I)c1]

thyroxine methyl ester,

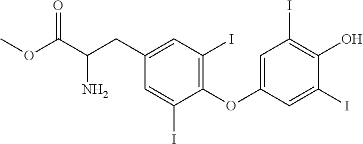 [SMILES: COC(=O)C(N)Cc1cc(I)c(Oc2cc(I)c(O)c(I)c2)c(I)c1]

and 6-iodo-L-DOPA methyl ester, among others. Although methyl esters are shown, higher alkyl esters may be employed.

In a particular embodiment shown in FIG. 7, thyroxine methyl ester (712) is coupled to a multi-arm polymer having carboxylic acid end groups (710) (only a single arm is illustrated) via an amide coupling reaction in the presence an amide coupling agent to form a multi-arm polymer (714) having thyroxine methyl ester residues that are linked to the polymer arms through amide linkages.

In some embodiments, a hydroxyl containing iodinated species is coupled to a multi-arm polymer with carboxylic acid end groups via an ester coupling reaction. Such reactions may be performed in the presence of a suitable ester coupling agent. Examples of suitable ester coupling agents include carbodiimide coupling agents such as those listed above. Particular examples of hydroxyl containing iodinated species, several of which are commercially available, include the following, among others: iopromide,

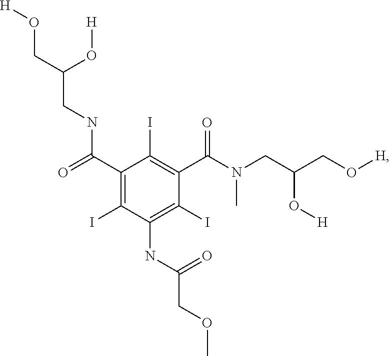

iopamidol,

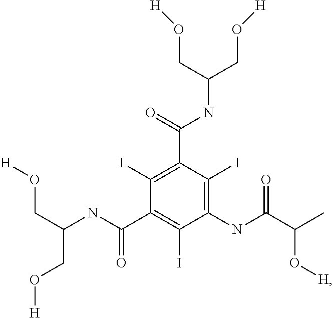

iomeprol,

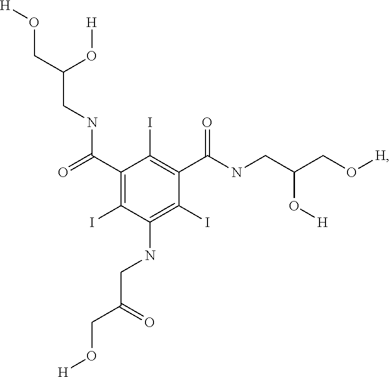

ioversol,

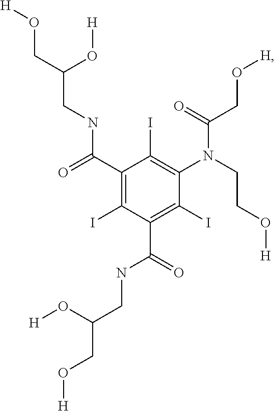

iobitridol,

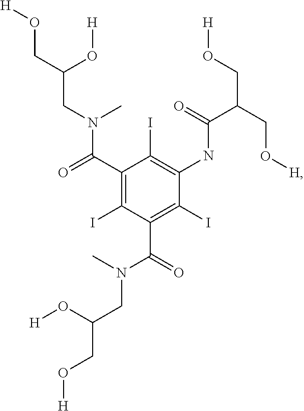

iotrolan,

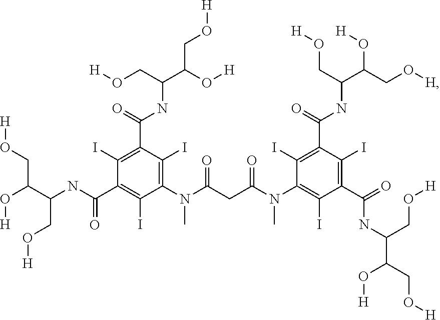

iodixanol,

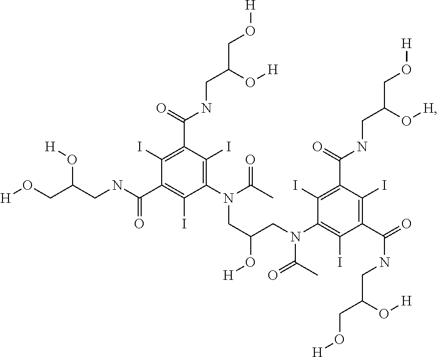

[3,5-bis(hydroxymethyl)-2,4,6-triiodo-phenyl]methanol,

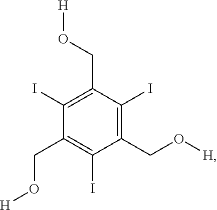

1 2,3,5,6-tetraiodo-1,4-benzenedimethanol,

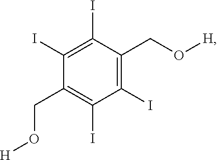

and 1-[2,4,6-triiodo-3,5-bis(1,2,3-trihydroxypropyl)phenyl]propane-1,2,3-triol,

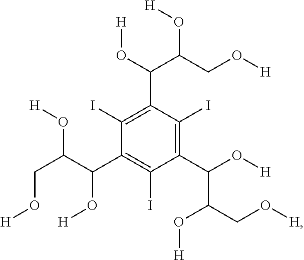

among others. Further examples of hydroxyl containing iodinated species can be selected from the iodinated polyols listed above.

For iodinated species that have more than one hydroxyl group, a significant molar excess of the iodinated species can be used during the coupling reactions ensure that only one of the hydroxyl groups of the iodinated species is attached to a polymer arm for a majority of the reacted iodinated species. The product can be further purified via chromatography or fractional crystallization methods.

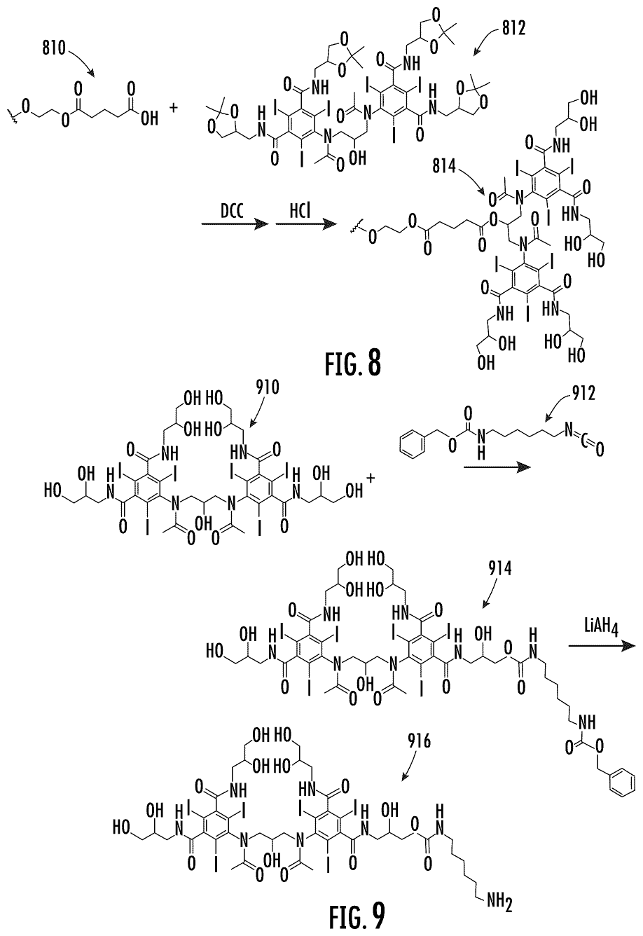

Several of these compounds contain vicinal diol groups which may be protected with 2,2-dimethoxypropane, formaldehyde or acetaldehyde to obtain a partially-acetal-protected polyiodinated aromatic compound. In a particular example, a vicinal-diol-containing iodinated species having plurality of vicinal diol groups, specifically, iodixanol is partially protected using 2, 2-dimethoxypropane in the presence of an organic solvent such as dimethylformamide and p-toluenesulfonic acid (PTSA) to obtain acetal-protected iodixanol in which a single hydroxyl group remains unprotected. With reference to FIG. 8, acetal-protected iodixanol (812) can be coupled to a multi-arm polymer with carboxylic acid end groups (810) via an ester coupling reaction in the presence of an ester coupling agent, DCC, followed by deprotection in HCl, to form a multi-arm polymer (814) having end groups that comprise iodixanol residues that are linked to the polymer arms through ester linkages.

In some embodiments, a hydroxyl-group containing iodinated species is reacted with a linker molecule to form an amine-containing group at a site of a hydroxyl group.

In some of these embodiments, a hydroxyl-group containing iodinated species is reacted with a protected isocyanate-containing amine compound that contains an isocyanate group and a protected amino group, for example, a protected isocyanate-substituted C₂-C₁₀-amine compound, followed by deprotection, to form an aminoalkyl carbamate group, for example, an amino-C₂-C₁₀-alkyl carbamate group, at a site of a hydroxyl group of the hydroxyl-group containing iodinated species. The amino group of this compound can then be used to couple the compound to carboxylic acid end groups of a multi-arm polymer via an amide coupling reaction as described above.

With reference to FIG. 9, a protected isocyanate-containing amine compound that contains an isocyanate group and a protected amino group for example, a Cbz-protected isocyanate-containing amine compound such as benzyl N-(6-isocyanatohexyl)carbamate (CAS #16644-46-5) (912), can be reacted with a hydroxyl-group-containing iodinated species, specifically, iodixanol (910), to form an intermediate product (914) that comprises a residue of the protected isocyanate-containing amine compound that is linked to a residue of the hydroxyl-group containing iodinated compound through a carbamate group. Once formed, the protected amino group in the intermediate product (914) can be deprotected with, for example, lithium aluminum hydride (LiAlH₄), to yield an amino-group containing iodinated compound (916) that comprises an amino-alkyl group that is linked to a residue of a hydroxyl-group containing iodinated compound through a carbamate group, more specifically an aminohexyl group that is linked to a residue of iodixanol through a carbamate group. The amino group of this compound (916) can then be used to couple the compound to carboxylic acid end groups of a multi-arm polymer via an amide coupling reaction as described above. Other examples of protected isocyanate-containing amine compound include tert-butyl N-(2-Isocyanatoethyl)carbamate (CAS #284049-22-5) and tert-butyl N-(3-isocyanatopropyl)carbamate (CAS #76197-73-4).

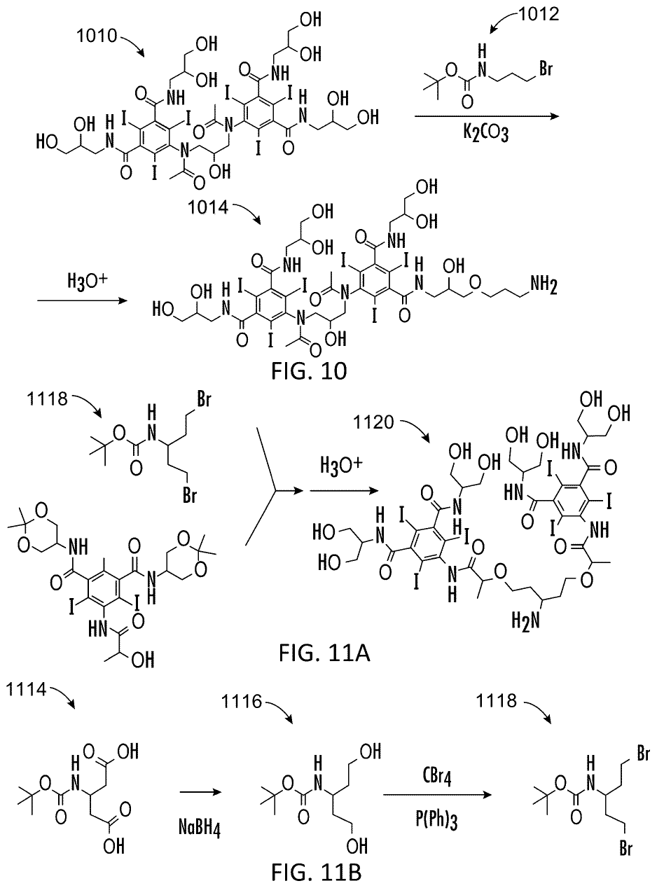

In some embodiments, an amino-group-containing iodinated species may be formed from a hydroxyl-group-containing iodinated species by reacting a hydroxyl group of the hydroxyl-group-containing iodinated species, with an amine-protected halogen-containing amino compound, for example, a halogen-containing amino compound that comprises an alkyl portion substituted by an amino group and one or more halide groups. For example, with reference to FIG. 10, a hydroxyl-group-containing iodinated species, specifically, iodixanol (1010), may be reacted with an amine-protected halogen-containing amino compound, specifically, Boc-protected 3-bromo-propylamine (1012), which comprises propyl core substituted with an amino group and a bromo group, in the presence of a strong base, such as potassium carbonate, followed by removal of the Boc protection, for example, by exposure to an acid such as HCl or trifluoroacetic acid, to yield an iodinated amino compound (1014) in which a residue of the hydroxyalkyl-substituted iodinated aromatic compound is linked to an amino alkyl group through an ether linkage. The amino group of this compound can then be used to couple the compound to carboxylic acid end groups of a multi-arm polymer via an amide coupling reaction as described above.

As another example, with reference to FIG. 11A, a hydroxyl-group-containing iodinated species, specifically, acetal-protected iopamidol (1112), may be reacted with an amine-protected, halogen-substituted aminoalkyl compound, specifically, tert-butyl N-[3-bromo-1-(2-bromoethyl)propyl]carbamate (1118), in the presence of a strong base, such as potassium carbonate followed by removal of the boc protection, for example, by exposure to an acid such as HCl or trifluoroacetic acid, to yield an amino-group-containing iodinated species (1120) in which two residues of the hydroxyalkyl-substituted iodinated aromatic compound are linked to an aminoalkyl group through two ether linkages. The amino group of this compound can then be used to couple the compound to carboxylic acid end groups of a multi-arm polymer via an amide coupling reaction as described above.

One method of forming the tert-butyl N-[3-bromo-1-(2-bromoethyl)propyl]carbamate (1118) of FIG. 11A is shown in FIG. 11B. With reference to FIG. 11B, an amine-protected polycarboxylic-acid-substituted aminoalkyl compound, specifically, 3-tert-butoxycarbonylaminopentanedioic acid (1114), is treated with a reducing agent such as sodium borohydride to reduce the carboxylic acid groups of the Boc-protected 3-aminopentane, 1,5-dicarboxylic acid (1114) to hydroxymethyl groups, thereby forming an amine-protected polyhydroxy-substituted aminoalkyl compound, specifically, tert-butyl N-[3-hydroxy-1-(2-hydroxyethyl)propyl]carbamate (1116). The hydroxyl groups are then converted to bromine groups using triphenylphosphine (P(Ph)₃) and carbon tetrabromide (CBr₄) as a source of bromine atoms, thereby providing a Boc-protected halogen-substituted aminoalkyl compound, specifically, Boc-protected 1,5-dibromo-3-aminopentane.

In other aspects, the present disclosure provides systems that comprise (a) a reactive polymer comprising a plurality of hydrophilic polymer segments and a plurality of first reactive groups, (b) a reactive multifunctional compound comprising a plurality of second reactive groups that are reactive with the first reactive groups, and (c) an iodinated multi-arm polymer as described above.

In various embodiments, the system is configured to deliver the reactive polymer, the reactive multifunctional compound, and the iodinated multi-arm polymer under conditions such that covalent crosslinks are formed between the first reactive groups of the reactive polymer and the second reactive groups of the reactive multifunctional compound, and a hydrogel is formed in which the iodinated multi-arm polymer is entrapped in the hydrogel. As a result, the diffusion of the iodinated multi-arm polymer from the radiopaque hydrogel is restricted, at least so long as the crosslinked reaction product and the iodinated multi-arm polymer remain intact.

In some aspects of the present disclosure, radiopaque hydrogels are provided that comprise (a) a crosslinked reaction product of a reactive polymer comprising a plurality of hydrophilic polymer segments and a plurality of first reactive groups and a reactive multifunctional compound comprising a plurality of second reactive groups that react with the first reactive groups such that covalent bonds are formed, and (b) an iodinated multi-arm polymer as described above. By crosslinking the reactive polymer with the reactive multifunctional compound in the presence of the iodinated multi-arm polymer, the iodinated multi-arm polymer becomes entrapped in the crosslinked reaction product. As a result, the diffusion of the iodinated multi-arm polymer from the radiopaque hydrogel is restricted, at least so long as the crosslinked reaction product and the iodinated multi-arm polymer remain intact.

As used herein, a “hydrogel” is a crosslinked polymer that contains water or can absorb water but does not dissolve when placed in water.

In various embodiments, the reactive polymer is a reactive multi-arm polymer that comprises a plurality of polymer arms linked to a core region, where the polymer arms comprise a hydrophilic polymer segment. One end of the hydrophilic polymer segment is covalently attached to the core region through a suitable linkage, and a first reactive group is covalently attached to an opposite end of the hydrophilic polymer segment through a suitable linkage.

Reactive multi-arm polymers in accordance with the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

Suitable first reactive groups include those that comprise electrophilic groups. Electrophilic groups may be selected, for example, from cyclic imide ester groups, such as succinimide ester groups,

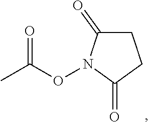

maleimide ester groups, glutarimide ester groups, diglycolimide ester groups, phthalimide ester groups, and bicyclo[2.2.1]hept-5-ene-2,3-dicarboxylic acid imide ester groups,

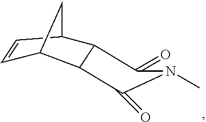

imidazole ester groups, imidazole carboxylate groups and benzotriazole ester groups, among other possibilities.

The electrophilic groups may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others. In various embodiments, the linking moiety comprises a hydrolysable ester group.

Suitable hydrophilic polymer segments can be selected from any of those described above in conjunction with the iodinated multi-arm polymers of the present disclosure.

In various embodiments, the core region comprises a residue of a polyol which is used to form the polymer arms. Suitable polyols can be selected from any of those described above in conjunction with the iodinated multi-arm polymers of the present disclosure.

Reactive multi-arm polymers in accordance with the present disclosure can be formed from carboxyl-terminated multi-arm polymers having arms that comprise one or more carboxyl end groups. Suitable carboxyl-terminated multi-arm polymers can be selected from any of those described above in conjunction with the iodinated multi-arm polymers of the present disclosure.

In various embodiments, an electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with a carboxylic-acid-terminated multi-arm hydrophilic polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form an activated ester end group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.).

As described above, terminal hydroxyl groups of a hydroxyl-terminated multi-arm polymer may be reacted with a cyclic anhydride compound (e.g., glutaric anhydride, succinic anhydride, malonic anhydride, adipic anhydride, diglycolic anhydride, 1,3-acetonedicarboxylic acid anhydride, etc.) to form carboxylic-acid-terminated polymer arms such as glutaric-acid-ester-terminated polymer arms, succinic-acid-ester-terminated polymer arms, malonic-acid-ester-terminated polymer arms, adipic-acid-ester-terminated polymer arms, diglycolic-acid-ester-terminated polymer arms, 1,3-acetonedicarboxylic-acid-ester-terminated polymer arms, and so forth. Reaction of an N-hydroxy cyclic imide compound with such a carboxylic-acid-terminated multi-arm polymer in the presence of a suitable coupling agent will result in a reactive multi-arm polymer in which cyclic imide ester groups are linked to the polymer arms through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

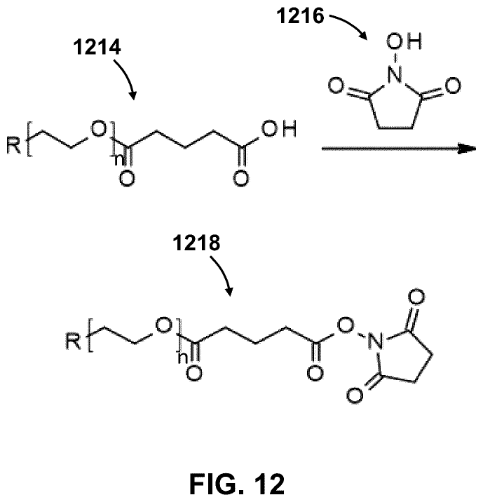

In a particular embodiment shown in FIG. 12, a carboxylic-acid-terminated polymer, specifically, glutaric-acid-terminated multi-arm PEO 1214, is reacted with N-hydroxysuccinimide 1216 in the presence of a suitable amide coupling agent, such as a carbodiimide coupling agent, to form a succinimidyl-glutarate-terminated multi-arm PEO 1218. Although a specific example using multi-arm PEO is illustrated, the preceding strategy is widely applicable to multi-arm polymers having hydrophilic polymer segments other PEO segments, such as the segments disclosed above. Some succinimidyl-glutarate-terminated multi-arm polymers are also available commercially. For example, succinimidyl-glutarate-terminated four-arm PEG and succinimidyl-glutarate-terminated eight-arm PEG are available from JenKem Technology USA. In some embodiments, the multifunctional compound is a compound having at two or more second reactive groups, for example, having 2, 3, 4, 5, 6, 7, 8, 9, 10, 11, 12, 13, 14, 15, 16, 17, 18, 19, 20 or more second reactive groups.

Suitable second reactive groups include nucleophilic groups. Specific nucleophilic groups include amino groups and thiol groups.

Some specific examples of such multifunctional compounds include, for example, polyamines that contain at two or more amino (—NH₂) groups (e.g., 2, 3, 4, 5, 6, 7, 8, 9, 10 or more amino groups in some embodiments), also referred to herein as polyamino compounds. Polyamino compounds suitable for use in the present disclosure include polyamino compounds that comprise two or more primary amine groups, for example, —(CH₂)ₓ—NH₂ groups where x is 1, 2, 3, 4, 5 or 6. Polyamino compounds suitable for use in the present disclosure include polyamino compounds that comprise two or more basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further examples of polyamino compounds which may be used as the multifunctional compound include ethylenediamine, ethylenetriamine, diethylene triamine, hexamethylenetriiamine, di(heptamethylene) triamine, di(trimethylene) triamine, bis(hexamethylene) triamine, tris(2-aminoethyl)amine, tris(3-aminopropyl)amine, 1,3,5-tris-(2-aminoethyl)-[1,3,5]triazinane-2,4,6-trione, N,N,N′-tris(2-aminoethyl)ethylenediamine, triethylene tetramine, tripropylene tetramine, N,N′,N′-tetrakis(2-aminoethyl)-1,2-ethanediamine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Compounds having at least two amino groups may also be made from polyols such those described above. For example, hydroxyl groups of a polyol maybe reacted in an ester coupling reaction in the presence of a suitable coupling agent with a carboxyl group of a suitable amino acid compound in which the amino group of the amino acid is protected with a suitable protective group (e.g., a tert-butyloxycarbonyl (tBoc) protective group). Examples of amino acids may be selected, for example, from beta amino acids such as 3-aminopropanoic acid (also known as beta-alanine), gamma amino acids such as 4-aminobutanoic acid (also known as gamma-aminobutyric acid, or GABA), delta amino acids such as 5-aminopentanoic acid, epsilon amino acids such as 6-aminohexanoic acid, etc. Deprotection of the protective groups results in a compound in which a primary amine group is linked to a residue of the polyol through a hydrolysable ester group at the site of each of the hydroxyl groups of the polyol.

As noted above, in some aspects, the present disclosure provides hydrogels that comprise an iodinated multi-arm polymer and a crosslinked reaction product of a reactive polymer comprising first reactive groups and a multifunctional compound comprising second reactive groups. Although the present disclosure provides specific examples where the first reactive groups comprise electrophilic groups and the second reactive groups comprise nucleophilic groups. Other pairs of reactive groups are also contemplated including the following, among others: (a) first reactive groups that comprise nucleophilic groups and second reactive groups that comprise electrophilic groups, (b) first reactive groups that comprise strained alkyne groups and second reactive groups that comprise azide groups, (c) first reactive groups that comprise azide groups and second reactive groups that comprise strained alkyne groups, (d) first reactive groups that comprise strained alkene groups and second reactive groups that comprise tetrazine groups, or (e) first reactive groups that comprise tetrazine groups and second reactive groups that comprise strained alkene groups.

In various embodiments, the hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values) for example, when measured on a bench-top micro CT system such as Xtreme CT from Scanco Medical (Wangen-Brüttisellen, Switzerland) or similar.

The hydrogels of the present disclosure can be used in a variety of biomedical applications, including implants, medical devices, and pharmaceutical compositions.

In some aspects of the present disclosure, a system is provided that comprises (a) a first composition that comprises a reactive polymer as described herein and (b) a second composition that comprises multifunctional compound as described herein, wherein the first composition, the second composition, or both, comprises an iodinated multi-arm polymer as described herein, and wherein when the first and second compositions are combined, covalent crosslinks form between the reactive polymer and the multifunctional compound, creating a hydrogel and trapping the iodinated multi-arm polymer in the hydrogel.

The first composition may be a first fluid composition comprising the multifunctional compound or a first dry composition that comprises the multifunctional compound, to which a suitable fluid such as water for injection, saline, etc. can be added to form a first fluid composition. In addition to the multifunctional compound, the first composition may further comprise an iodinated multi-arm polymer and/or additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise an iodinated multi-arm polymer and/or additional agents, including therapeutic agents, imaging agents, colorants, tonicity adjusting agents, suspension agents, wetting agents, and pH adjusting agents as described below.

In some embodiments, the system is configured to combine a first fluid composition comprising the multifunctional compound with a second fluid comprising the reactive polymer, wherein the first composition, the second composition, or both, comprises an iodinated multi-arm polymer as described herein, and wherein the first composition, the second composition, or both, may optionally comprise additional agents as described herein. Upon mixing the first and second fluid compositions, the multifunctional compound crosslink with the reactive polymer, forming a hydrogel and trapping the iodinated multi-arm polymer in the hydrogel. The first and second fluid compositions may be combined to form radiopaque crosslinked hydrogels, either in vivo or ex vivo.

In some embodiments, the multifunctional compound is initially combined with the reactive polymer under conditions where crosslinking between the reactive polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multifunctional compound and the reactive polymer, thereby forming a crosslinked product.

In some embodiments, the system comprises (a) a first composition that comprises a multifunctional compound as described herein and a reactive polymer as described herein and (b) a second composition, specifically, an accelerant composition, that contains an accelerant that is configured to accelerate a crosslinking reaction between the multifunctional compound and the reactive polymer. In some embodiments, the first composition may further comprise an iodinated multi-arm polymer and/or additional agents as described herein. In some embodiments, the second composition may further comprise an iodinated multi-arm polymer and/or additional agents as described herein. In some embodiments, the system may further comprise a third composition that comprises an iodinated multi-arm polymer as described herein.

In some embodiments, the system comprises (a) a first composition that comprises multifunctional compound as described herein, (b) a second composition that comprises a reactive polymer as described herein, and (c) a third composition, specifically, an accelerant composition, that contains an accelerant that is configured to accelerate a crosslinking reaction between the multifunctional compound and the reactive polymer. In some embodiments, the first composition may further comprise an iodinated multi-arm polymer and/or additional agents as described herein. In some embodiments, the second composition may further comprise an iodinated multi-arm polymer and/or additional agents as described herein. In some embodiments, the third composition may further comprise an iodinated multi-arm polymer and/or additional agents as described herein. In some embodiments, the system may further comprise a fourth composition that comprises an iodinated multi-arm polymer as described herein.

The first composition may be a first fluid composition comprising the multifunctional compound that is buffered to an acidic pH or a first dry composition that comprises the multifunctional compound, to which a suitable fluid such as water for injection, saline, an acidic buffer solution, etc. can be added to form a first fluid composition comprising the multifunctional compound that is buffered to an acidic pH. In some embodiments, for example, the acidic buffering composition may comprise monobasic sodium phosphate, among other possibilities. The first fluid composition comprising the multifunctional compound may have a pH ranging, for example, from about 3 to about 5. In addition to the multifunctional compound, the first composition may further comprise an iodinated multi-arm polymer and/or additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the multifunctional compound that is buffered to an acidic pH. In addition to the reactive polymer, the second composition may further comprise an iodinated multi-arm polymer and/or additional agents, including therapeutic agents, imaging agents, colorants, tonicity adjusting agents, suspension agents, wetting agents, and pH adjusting agents as described below.

In a particular embodiment, the first composition is a first fluid composition comprising the multifunctional compound (as well as an iodinated multi-arm polymer and/or additional agents in some cases) that is buffered to an acidic pH and the second composition is a dry composition that comprises the reactive polymer (as well as an iodinated multi-arm polymer and/or additional agents in some cases). The first composition may then be mixed with the second composition to provide a prepared fluid composition that is buffered to an acidic pH and comprises the multifunctional compound and the reactive polymer. In a particular example, a syringe may be provided that contains the first fluid composition comprising the multifunctional compound that is buffered to an acidic pH, and a vial may be provided that comprises the second dry composition (e.g., a powder) that comprises the reactive polymer. The syringe may then be used to inject the first fluid composition into the vial containing the reactive polymer to form a prepared fluid composition that is buffered to an acidic pH and contains the multifunctional compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise an iodinated multi-arm polymer and/or additional, including those described below.

A prepared fluid composition that is buffered to an acidic pH and comprises the multifunctional compound and the reactive polymer as described above (as well as an iodinated multi-arm polymer and/or additional agents in some cases) and a fluid accelerant composition that is buffered to basic pH as described above (which may include an iodinated multi-arm polymer and/or additional agents in some cases) may be combined form radiopaque crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises multifunctional compound as described above and a second reservoir that contains a second fluid composition that comprises a reactive polymer as described above, wherein the first and second fluid compositions form a crosslinked product upon mixing. As previously noted, the first fluid composition, the second fluid composition, or both, comprises an iodinated multi-arm polymer as described herein. Moreover, the first fluid composition, the second fluid composition, or both, may optionally comprise additional agents as described herein.

In some embodiments, the system may include a delivery device that comprises a first reservoir that contains a first fluid composition that comprises the multifunctional compound and the reactive polymer and is buffered to an acidic pH, such as the prepared fluid composition previously described, and a second reservoir that contains second fluid composition, such as the fluid accelerant composition previously described. As previously noted, the first fluid composition, the second fluid composition, or both, comprise an iodinated multi-arm polymer as described herein. Moreover, the first fluid composition, the second fluid composition, or both, may optionally comprise additional agents as described herein.

In either case, during operation, the first fluid composition and second fluid composition are dispensed from the first and second reservoirs and combined, whereupon the multifunctional compound and the reactive polymer and crosslink with one another to form a radiopaque crosslinked hydrogel.

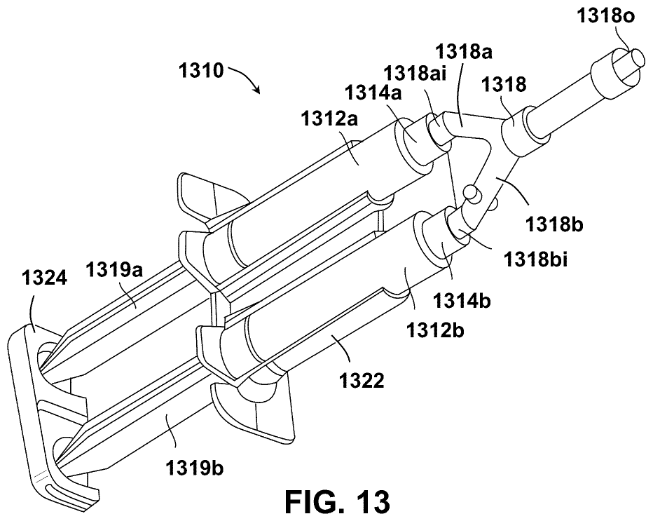

In particular embodiments, and with reference to FIG. 13, the system may include a delivery device 1310 that comprises a double-barrel syringe, which includes a first barrel 1312a having a first barrel outlet 1314a, which first barrel contains a first fluid composition as described above, a first plunger 1319a that is movable in the first barrel 1312a, a second barrel 1312b having a second barrel outlet 1314b, which second barrel 1312b contains a second fluid composition as described above, and a second plunger 1319b that is movable in the second barrel 1312b. In some embodiments, the device 1310 may further comprise a mixing section 1318 having a first mixing section inlet 1318ai in fluid communication with the first barrel outlet 1314a, a second mixing section inlet 1318bi in fluid communication with the second barrel outlet 1314b, and a mixing section outlet 1318o. Also shown are a syringe holder 1322 configured to hold the first and second syringe barrels 1312a, 1312b, in a fixed relationship and a plunger cap 1324 configured to hold the first and second plungers 1319a, 1319b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a radiopaque crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

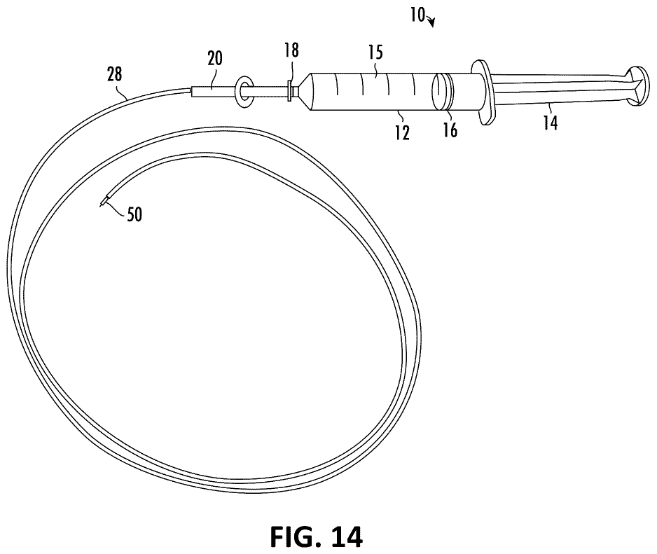

FIG. 14 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

During and/or after administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system comprising (a) an iodinated multi-arm polymer comprising first hydrophilic polymer segments and iodinated end groups, the iodinated multi-arm polymer comprising a first core region and three or more polymer arms linked to the first core region, each of the polymer arms comprising a first hydrophilic polymer segment and an iodinated end group, (b) a reactive multi-arm polymer comprising second hydrophilic polymer segments and first reactive groups, the reactive multi-arm polymer comprising a second core region and three or more polymer arms linked to the second core region, each of the polymer arms comprising a second hydrophilic polymer segment and a first reactive group and (c) a reactive multifunctional compound comprising a plurality of second reactive groups, the second reactive groups forming covalent bonds with the first reactive groups.

2. The system of claim 1, wherein the iodinated end group comprises at least one iodine-substituted monocyclic or multicyclic aromatic group.

3. The system of claim 2, wherein the iodine-substituted monocyclic or multicyclic aromatic group is substituted with one, two, three, four, five, six or more iodine atoms.

4. The system of claim 3, wherein the iodine-substituted monocyclic or multicyclic aromatic group is further substituted with one or more hydrophilic groups that are directly linked to the iodine-substituted monocyclic or multicyclic aromatic group or are linked to the iodine-substituted monocyclic or multicyclic aromatic group through a linking moiety that comprises amide group, an ether group, ester group, a carbamate group or a combination thereof.

5. The system of claim 4, wherein the hydrophilic groups are selected from acetamido groups and hydroxyl-containing groups.

6. The system of claim 2, wherein the iodine-substituted monocyclic or multicyclic aromatic group is linked to the first hydrophilic segment through a linkage that comprises an amide group, an ether group, an ester group, a carbamate groups or a combination thereof.

7. The system of claim 6, wherein the iodine-substituted monocyclic or multicyclic aromatic group is linked to the first hydrophilic segment through a linkage that comprises an amide group and an ester group.

8. The system of claim 6, wherein the iodine-substituted monocyclic or multicyclic aromatic group is linked to the first hydrophilic segment through a linkage that comprises two ester groups.

9. The system of claim 6, wherein the iodine-substituted monocyclic or multicyclic aromatic group is linked to the first hydrophilic segment through a linkage that comprises an amide group and a carbamate group.

10. The system of claim 6, wherein the iodine-substituted monocyclic or multicyclic aromatic group is linked to the first hydrophilic segment through a linkage that comprises an amide group and an ether group.

11. The system of claim 1, wherein the first core region, the second core region, or the first and second core regions comprise a polyol residue and wherein the first hydrophilic polymer segments are optionally linked to the first core region through a hydrolysable ester.

12. The system of claim 1, wherein the first hydrophilic polymer segments, the second hydrophilic polymer segments, or both the first and the second hydrophilic polymer segments are selected from poly(alkylene oxide) segments, polysaccharide segments, polyoxazoline segments, polydioxanone segments, polypeptide segments, and polyvinyl alcohol segments.

13. The system of claim 1, wherein the first reactive end groups are linked to the second hydrophilic polymer segments through a linkage that comprises a hydrolysable ester or wherein the first reactive end groups are linked to the second hydrophilic polymer segments through a linkage that does not comprise a hydrolysable ester.

14. The system of claim 1, wherein the first reactive end groups are electrophilic groups.

15. The system of claim 1, wherein the second reactive groups are nucleophilic groups.

16. A method of treatment comprising administering to a subject a mixture that comprises (a) an iodinated multi-arm polymer comprising first hydrophilic polymer segments and iodinated end groups, the iodinated multi-arm polymer comprising a first core region and three or more polymer arms linked to the first core region, each of the polymer arms comprising a first hydrophilic polymer segment and an iodinated end group, (b) a reactive multi-arm polymer comprising second hydrophilic polymer segments and first reactive groups, the reactive multi-arm polymer comprising a second core region and three or more polymer arms linked to the second core region, each of the polymer arms comprising a second hydrophilic polymer segment and a first reactive group, and (c) a reactive multifunctional compound comprising a plurality of second reactive groups, the second reactive groups forming covalent bonds with the first reactive groups, under conditions such that the reactive multi-arm polymer and the reactive multifunctional compound crosslink after administration to form a hydrogel in which the iodinated multi-functional polymer is retained in the hydrogel via non-covalent interactions.

17. The method of claim 16, wherein the method comprises administering to the subject a first fluid composition that comprises the reactive multi-arm polymer and a second fluid composition that comprises the reactive multifunctional compound, wherein the iodinated multi-functional polymer is provided in the first fluid composition, the second composition, or both.

18. The method of claim 16, wherein the method comprises administering to the subject a first fluid composition that comprises the reactive multi-arm polymer and the reactive multifunctional compound and a second fluid composition that comprises an accelerant that accelerates formation of covalent crosslinks between the reactive multi-arm polymer and the reactive multifunctional compound, and wherein the iodinated multi-functional polymer is provided in the first fluid composition, the second composition, or both.

19. The method of claim 18, wherein the first fluid composition and the second fluid composition are delivered using a double barrel syringe.

20. A radiopaque crosslinked hydrogel composition comprising an iodinated multi-arm polymer comprising first hydrophilic polymer segments and iodinated end groups, the iodinated multi-arm polymer comprising a first core region and three or more polymer arms linked to the first core region, each of the polymer arms comprising a first hydrophilic polymer segment and an iodinated end group retained in a crosslinked reaction product of (a) a reactive multi-arm polymer comprising second hydrophilic polymer segments and first reactive groups, the reactive multi-arm polymer comprising a second core region and three or more polymer arms linked to the second core region, each of the polymer arms comprising a second hydrophilic polymer segment and a first reactive group and (b) a reactive multifunctional compound comprising a plurality of second reactive groups, the second reactive groups forming covalent bonds with the first reactive groups, under conditions such that the reactive multi-arm polymer and the reactive multifunctional compound crosslink after administration to form a hydrogel in which the iodinated multi-functional polymer is retained in the hydrogel via non-covalent interactions.
